(19) United States
(12) Patent Application Publication
(10) Pub. No.: US 2004/0267092 A1
(43) Pub. Date: Dec. 30, 2004

(54) Distal hood component

(75) Inventor: Kouta Ishibiki, Tokyo (JP)

Correspondence Address: Paul J. Esatto, Jr., Scully, Scott, Murphy & Presser, 400 Garden City Plaza, Garden City, NY, 11530, US

(73) Assignee: OLYMPUS CORPORATION, TOKYO (JP)

(21) Appl. No.: 10/899,645

(22) Filed: Jul. 27, 2004

Related U.S. Application Data
(63) Continuation of application No. PCT/JP03/02025, filed on Feb. 25, 2002.

(30) Foreign Application Priority Data
Feb. 25, 2002 (JP) 2002-048315

(51) Int. Cl. A61B 1/00

(52) U.S. Cl. 600/127

(57) ABSTRACT

In the present invention, a distal hood component 20 is detachably provided at the distal end 11 of an insertion portion 10 of an endoscope 1. The distal hood component 20 is formed from a soft, elastic soft material. The distal hood component 20 includes a protrusion 21 protruding from the distal end 11 and an endoscope fixation portion 22 into which the distal end 11 is fitted. Convex portions 24 and 24 and concave portions 23 and 23 are provided on the protrusion 21. The shapes of the convex portions 24 and 24 and concave portions 23 and 23 are adjusted in order that the convex portions 24 and 24 of the protrusion 21 are deformed by a force of 0.29 Mpa or less applied from the end of the protrusion 21.

This application claims benefit of Japanese Application No. 2002-048315 filed on Feb. 25, 2002, and PCT Application No. PCT/JP03/02025 filed in Japan on Feb. 25, 2003, the contents of which are incorporated by this reference.

### TECHNICAL FIELD

The present invention relates to a distal hood component provided at the distal end of an insertion portion of an endoscope.

### BACKGROUND ART

So far, an example of known endoscope apparatuses used for surgery is provided with an observational optical system, a light guide, an air and water feed hole and a suction hole at the distal end of an insertion portion of an endoscope. With respect to such an endoscope apparatus, light is applied to a subject, e.g., living body tissue, from the light guide, the subject irradiated with the light is visually identified through an objective lens, and air, water, or other substance fed from the air and water feed hole can be suctioned through the suction hole.

Furthermore, in some endoscopes, a hood is provided at the distal end of an insertion portion of an endoscope in order to ensure a closest approach distance between an observation window of an observational optical system and a subject.

Japanese Unexamined Patent Application Publication No. 2001-224550 discloses an endoscope having a substantially cylindrical hood as an example of endoscopes provided with a hood. With respect to the shape of the endoscope, at least one place of a circumferential wall of the hood is partially cut away at the position in the diagonal direction of the observational field of view.

On the other hand, Japanese Unexamined Patent Application Publication No. 59-93413 discloses a substantially cylindrical, pliable hood having a notch at the opening end of the hood.

With respect to the technology described in Japanese Unexamined Patent Application Publication No. 59-93413, a notch was provided in a part of the hood and, therefore, the hood was deformed by a force from the outside perimeter direction of the hood. However, deformation by a force from the end side of the hood was difficult because the hood was substantially cylindrical, and there have been problems similar to those of the technology described in Japanese Unexamined Patent Application Publication No. 2001-224550.

When the strength of the hood is increased, the surgeon must carefully perform an operation during insertion into a body cavity in order that the patient is allowed to have no uncomfortable feeling. However, the amount of force was not taken into consideration while the force was applied from the end side of the hood and the hood was thereby deformed. Consequently, there was a possibility that the patient might have an uncomfortable feeling.

### DISCLOSURE OF INVENTION

The present invention is a distal hood component which is detachably provided at the distal end of an insertion portion of an endoscope or is integrally provided and which includes a protrusion protruding in the direction of the observational field of view of the endoscope, wherein the protrusion is made of an elastically deformable soft material, at least two concave portions are provided in order to allow the ranges of continuation of the convex portions of the protrusion to become about 180° or less in the circumferential direction, and the protrusion can be deformed by application of a force from the end side of the protrusion.

### BRIEF DESCRIPTION OF THE DRAWINGS

FIG. 1 to FIG. 5 relate to a first embodiment of the present invention. FIG. 1 is a perspective view of the distal end of an endoscope fitted with a distal hood component. FIG. 2 is a front view of the distal end of the endoscope. FIG. 3 is a diagram for illustrating a force applied to the distal hood component from a target. FIG. 4 is a diagram for illustrating deformation due to application of the force to the distal hood component. FIG. 5 is a diagram for illustrating end surfaces of convex portions of the distal hood component.

FIG. 6 to FIG. 8 relate to a second embodiment of the present invention. FIG. 6 is a front view of the distal end of an endoscope fitted with a distal hood component. FIG. 7 is a sectional view of the distal hood component. FIG. 8 is a diagram for illustrating deformation due to application of a force to the distal hood component.

FIG. 9 is a sectional view of a distal hood component fitted to an insertion portion of an endoscope according to a third embodiment of the present invention.

FIG. 10 is a sectional view of a distal hood component fitted to an insertion portion of an endoscope according to a fourth embodiment of the present invention.

FIG. 11 is a diagram for illustrating a screen display of a monitor according to a fifth embodiment of the present invention.

FIG. 12 to FIG. 14 relate to a sixth embodiment of the present invention. FIG. 12 is a sectional view of a distal hood component fitted to an insertion portion of an endoscope. FIG. 13 is a front view of the distal end of an endoscope fitted with a distal hood component. FIG. 14 is a diagram for illustrating a screen display of a monitor.

FIG. 15 and FIG. 16 relate to a seventh embodiment of the present invention. FIG. 15 is a sectional view of a distal hood component fitted to an insertion portion of an endoscope. FIG. 16 is a diagram for illustrating deformation of the distal hood component.

FIG. 17 is a front view of the distal end of an endoscope fitted with a distal hood component according to an eighth embodiment of the present invention.

FIG. 18 is a front view of the distal end of an endoscope fitted with a distal hood component according to a ninth embodiment of the present invention.

FIG. 19 is a front view of the distal end of an endoscope including a distal hood component according to a tenth embodiment of the present invention.

FIG. 20 is a front view of the distal end of an endoscope including a distal hood component according to an eleventh embodiment of the present invention.

### BEST MODE FOR CARRYING OUT THE INVENTION

In order to describe the present invention in further detail, this will be explained with reference to the attached drawings.

### FIRST EMBODIMENT

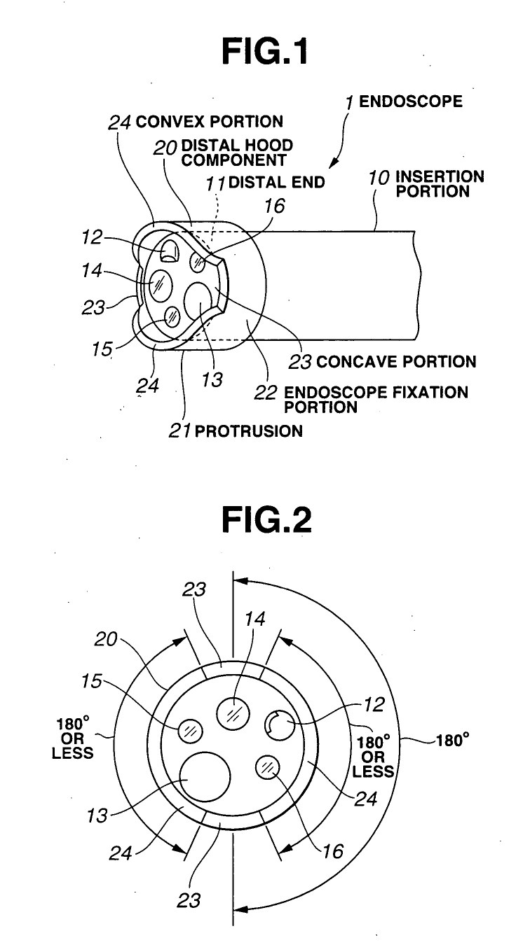

FIG. 1 to FIG. 5 show the first embodiment of the present invention.

It is an object of the first embodiment to provide a distal hood component without using an expensive material, wherein an observational field of view is ensured, breakage is prevented, and the patient is allowed to have no uncomfortable feeling.

As shown in FIG. 1, an endoscope 1 constitutes an endoscope apparatus together with a light source device, a video processor and a monitor, although not shown in the drawing. A distal hood component 20 is detachably provided at the distal end 11 of an insertion portion 10 of the endoscope 1. In this case, the distal hood component 20 is formed to have a substantially cylindrical shape, and is press-fitted into the distal end 11, and fixed thereto.

The distal hood component 20 is formed from a soft, elastic soft material, for example, vulcanized rubber, e.g., silicon rubber and fluororubber; thermoplastic elastomers, e.g., urethane-based elastomers, acrylic-based elastomers and olefin-based elastomers.

The distal hood component 20 includes a protrusion 21 protruding from the distal end 11 and an endoscope fixation portion 22 into which the distal end 11 is fitted. Two concave portions 23 and 23 are provided on the protrusion 21 in order that the protrusion 21 can be deformed by a force applied from the end of the protrusion 21. Two convex portions 24 and 24 are provided on the protrusion 21 resulting from the formation of the two concave portions 23 and 23.

As shown in FIG. 1 and FIG. 2, an air and water feed nozzle 12 which is a hole for feeding air and water, a suction hole 13, an observational optical system 14 and illumination windows 15 and 16 are provided on the end surface of the distal end 11.

The observational optical system 14 includes objective lenses, and a front-end lens of the objective lenses is arranged at the observation window. An image incident end surface (in the case of an electronic endoscope, an image pick up surface of a solid-state imaging device) of an image fiber bundle is arranged in the endoscope base end side of the objective lenses. An output end surface of the light guide fiber bundle is arranged inside the observation windows 15 and 16.

As shown in FIG. 2, the locations of the concave portions 23 and 23 are adjusted at the positions which allow the ranges of continuation of the convex portions 24 and 24 to become 180° or less on the circumference. In this case, the concave portions 23 and 23 are provided at intervals of 180° in the present embodiment.

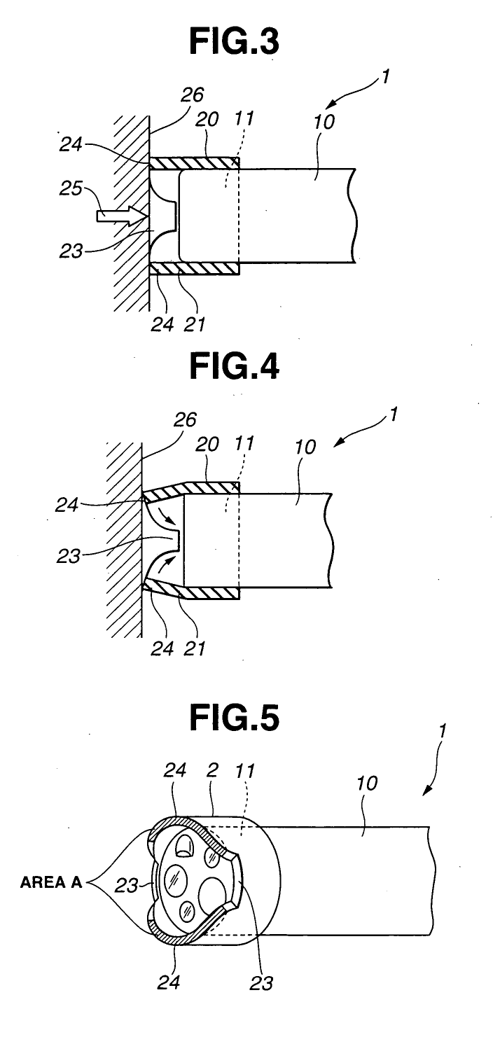

As shown in FIG. 3, the concave portions 23 and 23 are formed in order that the convex portions 24 and 24 are deformed as shown in FIG. 4 by a force of 0.29 Mpa or less when the force indicated by an arrow 25 is applied from a target 26 to the end of the protrusion 21.

Since the basic shape of the protrusion 21 is substantially cylindrical, deformation is unlikely to occur in the outside perimeter direction when a force is applied from the end side, but the deformation is likely to occur in the inside perimeter direction.

Here, as shown in FIG. 5, the area of the end surfaces of the convex portions 24 and 24 in contact with the target is represented by A (diagonally shaded portion), and a pressure applied to this diagonally shaded portion is represented by P.

As shown in FIG. 3, when a force is applied to the end surfaces of the convex portions 24 and 24, the applied force F can be determined by the following formula.

F=P×A (1)

Here, the convex portions 24 and 24 are assumed to be formed in order to deform at a pressure P of 0.2 Mpa (2 kgf/cm²).

For example, when A=0.4 cm², the shapes, sizes and materials of the convex portions 24 and 24 and the concave portions 23 and 23 of the distal hood component 20 are adjusted in order that the convex portions 24 and 24 are deformed at a force F=0.8 kgf based on the formula (1), while the force F is applied to the end surface of the convex portions 24 and 24.

For example, when A=0.3 cm², the distal hood component 20 is formed in order that the deformation occurs at an applied force F of F=0.6 kgf based on the formula (1).

In such a configuration, since the convex portions 24 and 24 are formed in order that the ranges of continuation become 180° or less on the circumference, the convex portions 24 and 24 are easily deformed in an inward direction of the protrusion 21 even when a force is applied from the end side of the protrusion 21.

In this manner, stress does not concentrate onto the protrusion 21 and the endoscope fixation portion 22, and breakage of the convex portions 24 and 24 can be sufficiently prevented even when an inexpensive material is used for the distal hood component 20. Furthermore, even when a simple structure is used, in which the endoscope fixation portion 22 of the distal hood component 20 is fitted to the distal end 11, the endoscope fixation portion 22 can be sufficiently prevented from deviating or detaching from the distal end 11.

On the other hand, a document, Yoshiharu Uno, “Saikei daichou naishikyou CF-SV no anzenseino kentou (Discussion on safety of slim colonoscope CF-SV)”, Japanese journal of medical instrumentation, vol. 67, No. 7 bessatsu (supplementary volume), issued on Jul. 1, 1997, p.289-292, discloses that application of a force of 3 to 4 kg/cm² or more to an intestinal paries causes perforation of the intestinal paries with a high possibility in theory.

Consequently, the surgeon operates the endoscope in order that a force of more than or equal to the above-described value is not applied to the intestinal paries. That is, the surgeon operates the endoscope in order that a force of 3 to 4 kg/cm² or more is not applied to the protrusion 21.

In the present embodiment, when the protrusion 21 is pressed against a mucosa during an inspection using the endoscope, the convex portions 24 and 24 are deformed at 0.2 Mpa. That is, deformation reliably occurs at about 0.29 Mpa or less (3 kgf/cm² or less). Since the surgeon performs operations in order that such a force is not applied, breakage of the protrusion 21 and the endoscope fixation portion 22 can be prevented from occurring.

Furthermore, when the protrusion 21 is pressed against a mucosa targeted for observation, the convex portions 24 and 24 are deformed, and the distance between the observational optical system and the observation target is thereby decreased compared with that in the natural state, so that the observation target is observed in a manner different from that in the ordinary state. Consequently, the surgeon can become aware that the convex portions 24 and 24 are deformed before a force of about 0.29 Mpa or more (3 kgf/cm² or more) is applied to the convex portions 24 and 24.

According to the present embodiment, since the distal hood component 20 is used, the observational field of view can be easily ensured, and an endoscope apparatus having excellent observational performance can be provided. Since the convex portions 24 and 24 are formed to deform by a force of 0.29 Mpa or less, application of an excessive force to the distal hood component 20 can be prevented. Therefore, breakage of the distal hood component 20 can be prevented from occurring, and the durability can be improved, without using an expensive material. Since the convex portions 24 and 24 are deformed by a force of 0.29 Mpa or less, the patient can be prevented from having an uncomfortable feeling. Furthermore, the surgeon can become aware that the convex portions 24 and 24 are deformed before a force of about 0.29 Mpa or more (3 kgf/cm² or more) is applied to the convex portions 24 and 24.

### SECOND EMBODIMENT

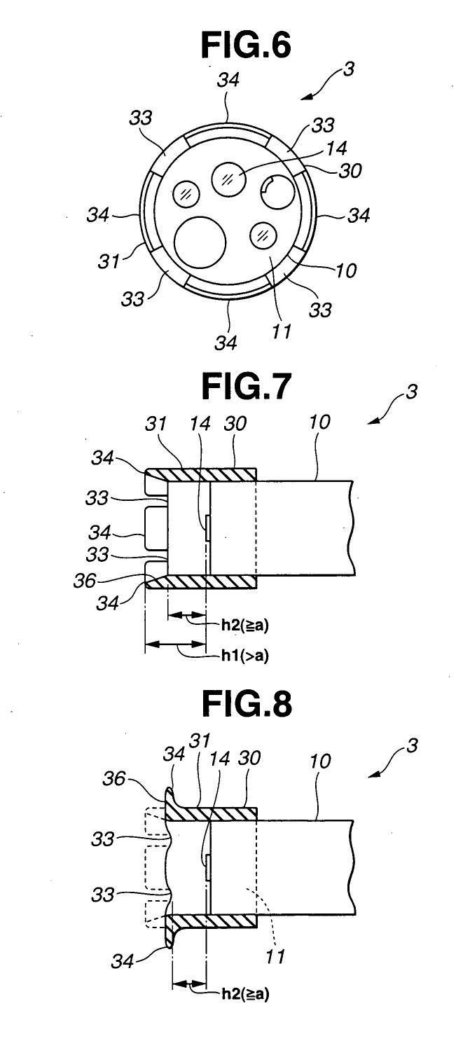

FIG. 6 to FIG. 8 show the second embodiment of the present invention.

As shown in FIG. 6, an endoscope 3 according to the present embodiment is different from the first embodiment shown in FIG. 2 only in a distal hood component 30, and an insertion portion 10 has a configuration similar to that in the first embodiment.

The distal hood component 30 is formed from a material similar to that for the distal hood component 20 in the first embodiment. Concave portions 33 are provided at four places on a protrusion 31 of the distal hood component 30 in order that the protrusion 31 can be deformed when a force is applied from the end of the protrusion 31.

In this case, four concave portions 33 are arranged at intervals of 90°. Since four concave portions 33 are provided, four convex portions 34 are provided on the protrusion 31.

As shown in FIG. 7, the depth of field of an observational optical system 14 is adjusted to be 3 mm to 100 mm. The protrusion length h1 of the convex portion 34 from a front-end lens of the observational optical system 14 is adjusted to be longer than the value of near point a=3 mm of the depth of field. For example, the protrusion length h1 is adjusted to be 5 mm.

The protrusion length h2 of the concave portion 33 is adjusted to be longer than or substantially equal to the value of near point a=3 mm of the depth of field. For example, the protrusion length h2 is adjusted to be 3 mm.

A taper portion 36 having the shape increasing in diameter with increasing proximity to the end side is provided on the inside perimeter surface of the convex portion 34.

In such a configuration, as shown in FIG. 8, the convex portion 34 is deformed to expand outward by the action of the taper portion 36 when a force is applied from the end side of the convex portion 34, and the amount of visible range of the convex portion 34 is not increased in the observational field of view. Consequently, the field of view is not blocked.

Since the range of the protrusion length h2 (=3 mm) of the concave portion 33 of the protrusion 31 is not changed even when the convex portion 34 is deformed, the distance between the observational optical system 14 and the observation target is kept at 3 mm or more, and the observation target does not fall out of focus.

As described above, according to the present embodiment, the observational field of view can be ensured, breakage can be prevented, and the patient is allowed to have no uncomfortable feeling without using an expensive material. In addition, an endoscope can be provided, which has a wide field of view, a clear field of view and excellent observational performance even when the convex portion 34 is deformed.

### THIRD EMBODIMENT

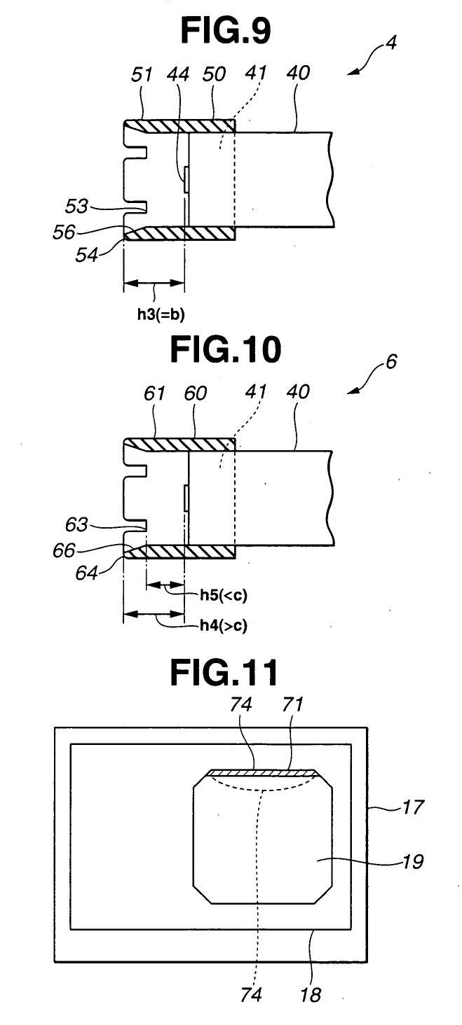

FIG. 9 shows the third embodiment of the present invention.

As shown in FIG. 9, in an endoscope 4 according to the present embodiment, the depth of field of an observational optical system 44 provided at the distal end 41 of an insertion portion 40 is adjusted to be 4 mm to 100 mm.

A distal hood component 50 is formed from a material similar to that for the distal hood component 20 in the first embodiment.

Concave portions 53 are provided at four places on a protrusion 51 of the distal hood component 50 at intervals of 90°. Since four concave portions 53 are provided, four convex portions 54 are provided on the protrusion 51. A taper portion 56 having the shape increasing in diameter with increasing proximity to the end side is provided on the inside perimeter surface of the convex portion 54.

The protrusion length h3 of the convex portion 54 from the observational optical system 44 is adjusted to be substantially the same as the value of near point b=4 mm of this depth of field.

In such a configuration, when the convex portion 54 is deformed during use of the endoscope 4, the distance between the observational optical system 44 and the observation target becomes smaller than the value of near point b of the depth of field of the observational optical system 44, and the observation image thereby falls out of focus. In this manner, the surgeon can become aware that the distal hood component 50 is deformed.

According to the present embodiment, effects similar to those in the second embodiment shown in FIG. 6 to FIG. 8 can be achieved and, in addition, the surgeon can further easily become aware that the distal hood component 50 is deformed.

### FOURTH EMBODIMENT

FIG. 10 shows the fourth embodiment of the present invention.

As shown in FIG. 10, in an endoscope 6 according to the present embodiment, an insertion portion 40 having the same depth of field as that shown in FIG. 9 is used. The distal hood component 60 is formed from a material similar to that for the distal hood component 20 in the first embodiment.

Four concave portions 63 are provided at four places on a protrusion 61 of the distal hood component 60 at intervals of 90°. According to this, four convex portions 64 are provided on the protrusion 61. A taper portion 66 having the shape increasing in diameter with increasing proximity to the end side is provided in the end side of the convex portion 64.

The protrusion length h4 of the convex portion 64 from the observational optical system 44 is adjusted to be longer than the value of near point c=4 mm of the depth of field of the observational optical system 44. For example, the protrusion length h4 is adjusted to be 6 mm.

On the other hand, the protrusion length h5 of the concave portion 63 is adjusted to be shorter than the value of near point c=4 mm of the depth of field. For example, the protrusion length h5 is adjusted to be 2 mm. With respect to this adjustment, the convex portion 64 can be deformed in order that the distance between the observational optical system and the target becomes smaller than the value of near point of the depth of field when a force is applied to the convex portion 64.

In such a configuration, when the convex portion 64 is deformed and the end of the convex portion 64 becomes shorter than the value of near point c=4 mm, the distance between the observational optical system and the observation target becomes smaller than the value of near point of the depth of field and, therefore, the observation image falls out of focus.

According to the present embodiment, effects similar to those in the second embodiment shown in FIG. 6 to FIG. 8 can be achieved and, in addition, the surgeon can further easily become aware that the hood is deformed more than necessary.

The present invention is not limited to the above-described first to fourth embodiments. The above-described protrusion may be formed to deform in an inward direction, and the above-described protrusion may be formed to deform in an outward direction.

### FIFTH EMBODIMENT

FIG. 11 shows the fifth embodiment of the present invention.

As shown in FIG. 11, a monitor 17 of an endoscope according to the present embodiment displays an observation image 19 at the right on a screen 18. The observation image 19 is square, rectangular or substantially tetragonal.

An endoscope apparatus according to the present embodiment is formed in order that at least a part of a convex portion 74 of a protrusion 71 overlaps the range of the observational field of view, so that the convex portion 74 of the protrusion 71 of the distal hood component can be seen in the observation image 19 on the monitor 17 in the natural state. The configuration of the endoscope except for this is similar to that in the first embodiment.

In such a configuration, when a force is applied from the target to the protrusion 71 of the distal hood component, the convex portion 74 of the protrusion 71 is deformed toward the inside perimeter side in a manner similar to that of the convex portion 24 shown in FIG. 4, and the deformation of the convex portion 74 can be recognized in the observation image 19 on the monitor 17, as indicated by a dotted line shown in FIG. 11.

According to the present embodiment, effects similar to those in the first embodiment shown in FIG. 1 to FIG. 5 can be achieved and, in addition, since the convex portion 74 is displayed in the observation image 19 on the monitor 17, the surgeon can further easily become aware that the distal hood component is deformed.

The present invention is not limited to the above-described fifth embodiment. The endoscope apparatus may be formed in order that when a force is applied to the protrusion 71 of the distal hood component, the protrusion 71 is deformed and a portion of the protrusion 71, at which a concave portion is provided, overlaps the range of the observational field of view or the intermediate portion between the convex portion 74 and the concave portion overlaps the range of the observational field of view. That is, in the present invention, the above-described protrusion is formed in order that the amount of the above-described protrusion visible in the observational field of view is increased when a part of the above-described protrusion is deformed.

### SIXTH EMBODIMENT

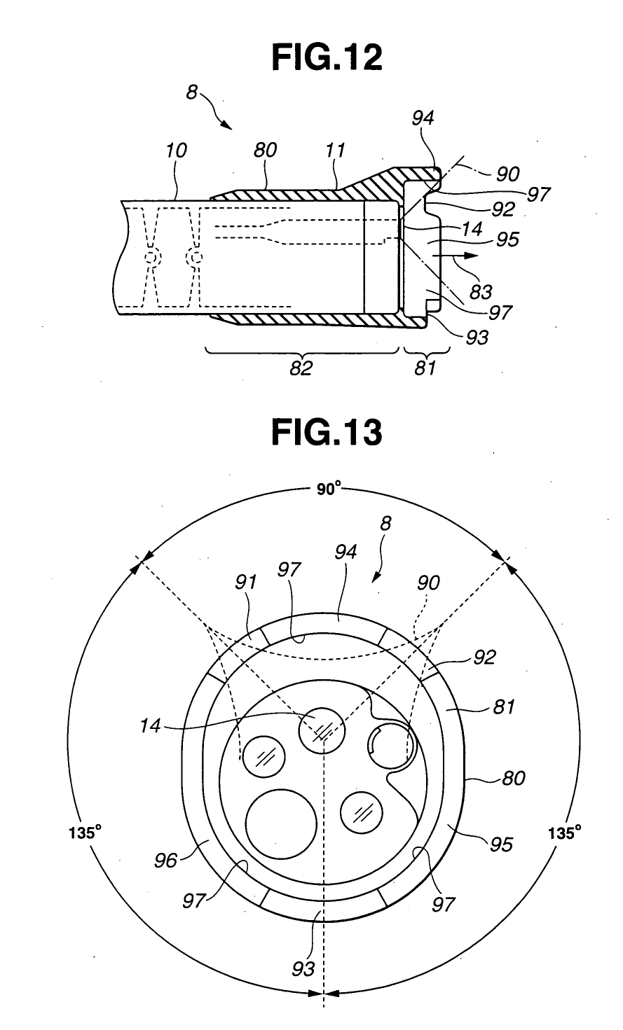

FIG. 12 to FIG. 14 show the sixth embodiment of the present invention.

As shown in FIG. 12, an endoscope 8 according to the present embodiment is different from the first embodiment shown in FIG. 2 only in the configuration of a distal hood component 80, and an insertion portion 10 has a configuration similar to that in the first embodiment.

The distal hood component 80 is formed from a material similar to that for the distal hood component 20 in the first embodiment. The distal hood component 80 includes a protrusion 81 protruding from a distal end 11 and an endoscope fixation portion 82 into which the distal end 11 is fitted.

Three concave portions 91, 92 and 93 are provided on the protrusion 81 of the distal hood component 80, as shown in FIG. 13, in order that the protrusion 81 can be deformed when a force is applied from the end of the protrusion 81. Since three concave portions 91, 92 and 93 are provided, three convex portions 94, 95 and 96 are provided on the protrusion 81.

Each of the concave portions 91 and 92 is formed to have a notch in the shape corresponding to the range 90 of the observational field of view of an observational optical system 14 shown in FIG. 12 and FIG. 13 in order that the protrusion 81 does not enter the range 90 of the observational field of view.

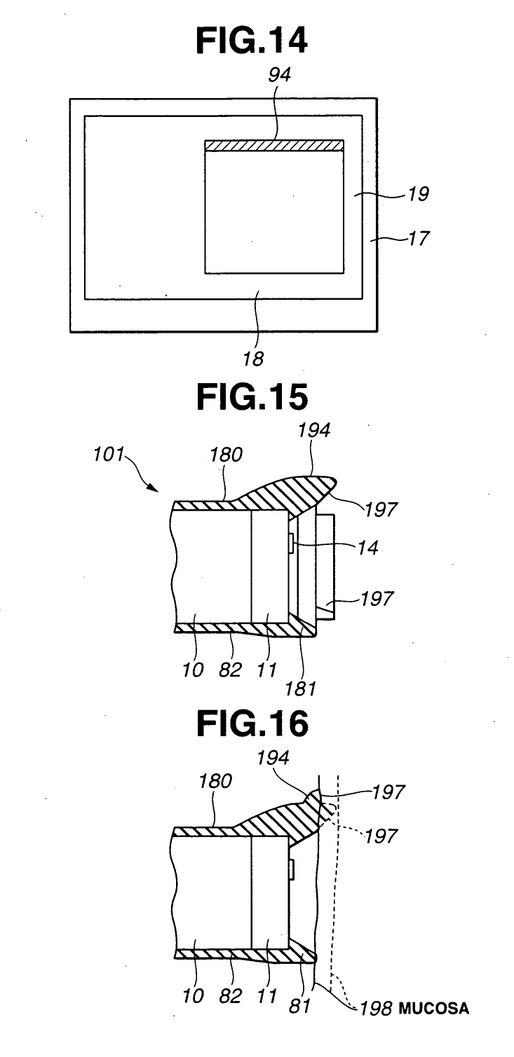

In this manner, the distal hood component 80 is configured in order that the protrusion 81 is not seen or hardly seen in the observation image 19 on a monitor 17 shown in FIG. 14 in the natural state. The concave portion 93 is provided in the location facing the convex portion 94 of the protrusion 81.

As shown in FIG. 13, the concave portion 91 and the concave portion 92 are arranged at an interval of about 90°. The concave portion 91 and the concave portion 93 are arranged at an interval of about 135°. The concave portion 92 and the concave portion 93 are arranged at an interval of about 135°.

Each of the convex portions 95 and 96 is formed in order that the range of continuation becomes 180° or less in the circumferential direction because of these intervals.

With respect to the distal hood component 80, the concave portions 91, 92 and 93 are provided in order that when a force is vertically applied from the end side of the protrusion to the end surface in a manner similar to that in the first embodiment, the convex portions 94, 95 and 96 begin to deform in an inward direction at a pressure of 0.29 Mpa or less. Interior surfaces 97 of the convex portions 94, 95 and 96 are formed to become substantially parallel to the insertion direction 83 of the endoscope shown in FIG. 12 in order to deform in an inward direction when a pressure is applied.

At least a part of the convex portions 94, 95 and 96, for example, the convex portion 94, is formed in the location close to a range 90 of the observational field of view in order that a part of the convex portion 94 enters the range 90 of the observational field of view by deformation thereof.

In such a configuration, when the protrusion 81 is pressed against a mucosa of an observation target and, thereby, the convex portion 94 is deformed, a part of the convex portion 94, which has not been seen, enters the range 90 of the observational field of view, and a part of the convex portion 94 becomes visible in the observation image 19 on the monitor 17.

According to the present embodiment, since the convex portion 94 of the protrusion 81 is deformed in an inward direction at a force of 0.29 Mpa or less, effects similar to those in the first embodiment shown in FIG. 1 to FIG. 5 can be achieved. In addition, since the convex portion 94 is displayed in the observation image 19 on the monitor 17 when the convex portion 94 is deformed, the surgeon can further easily become aware that the distal hood component is deformed.

The present invention is not limited to the above-described sixth embodiment. When a force is applied to the protrusion 81 of the distal hood component 80, the protrusion 81 may be deformed and a portion, at which a concave portion 91 is provided, may be seen in the observation image 19, or the intermediate portion between the convex portion 94 and the concave portion 91 may be seen. That is, in the present invention, the above-described protrusion is not visible in the observational field of view in the natural state, and a part of the above-described protrusion is visible in the observational field of view when the above-described protrusion is deformed.

### SEVENTH EMBODIMENT

FIG. 15 and FIG. 16 show the seventh embodiment of the present invention.

As shown in FIG. 15, an endoscope 101 according to the present embodiment is different from the sixth embodiment shown in FIG. 12 in that an inclined surface portion 197 is provided all around the inside perimeter surface side of a protrusion 181 of a distal hood component 180. An endoscope fixation portion 82 and an insertion portion 10 of the distal hood component 180 have configurations similar to those in the sixth embodiment.

The inclined surface portion 197 is in the shape increasing in diameter with increasing proximity to the end side of the protrusion 181. The convex portion 194 is formed to deform as shown in FIG. 16 at a force of 0.29 Mpa or less.

In such a configuration, as shown in FIG. 16, when the protrusion 181 is pressed against a mucosa 198 of an observation target and, therefore, a force is applied to the convex portion 194 from the end side, the convex portion 194 is deformed toward the outside perimeter side by the action of the inclined surface portion 197.

When the convex portion 194 is deformed, the inclined surface portion 197 contacts the mucosa 198 and, thereby, the contact area is increased. A pressure at the mucosa 198 in contact with the inclined surface portion 197 is decreased because of an increase in the contact area.

According to the present embodiment, since the convex portion 194 of the protrusion 181 is deformed outward at a force of 0.29 Mpa or less, the patient can be prevented from having an uncomfortable feeling, and effects similar to those in the second embodiment shown in FIG. 6 to FIG. 8 can be achieved. In addition, when the convex portion 194 is deformed, the inclined surface portion 197 contacts the mucosa and the contact area is increased. Consequently, application of an excessive pressure to the mucosa can be further prevented, and the patient can be further prevented from having an uncomfortable feeling.

The present invention is not limited to the above-described seventh embodiment. The inclined surface portion may be provided in at least one of the inside perimeter side and the outside perimeter side of the above-described protrusion in order that when the above-described convex portion 181 is pressed against a pressure surface and is thereby deformed, the contact area with the above-described pressure surface is increased.

In the first to the seventh embodiments, for example, the concave portion may not be protruded, and be flush with the end surface of the endoscope. Alternatively, the shape may have a concave portion from the end surface toward the base end portion side. Protrusion lengths of the plurality of concave portions may not be the same. All of the protrusion lengths of the convex portions may not be the same. The shape of the end portion of the convex portion itself may be a gently convex or concave shape, or may have fine concavities and convexities. It is essential that the whole protrusion of the hood component is formed from the concave portions and the convex portions.

Furthermore, the protrusions of the hood components shown in the first to the seventh embodiments may not be cylindrical, but may be tubular wherein the cross-sectional shape as the whole protrusion is elliptic or oval, has a partial linear portion, or is a polygonal shape, e.g., a substantially tetragonal shape and a substantially octagonal shape.

The protrusion is assumed to have a substantially tubular shape composed of a convex portion assembly. That is, the tubular shape having a cross section in the shape of combination of convex portions and concave portions is assumed, and the concave portions open toward at least the end portion of the protrusion may be provided in order that the ranges of continuation of portions, which are convex portions relative to the concave portions, become 180° or less in the circumferential direction while the cross section of the above-described tubular shape is approximated to the shape of an arc.

The distal hood component in each of the embodiments may be detachably provided at the distal end of the endoscope or may be integrally provided at the distal end of the endoscope while attachment and detachment cannot be performed.

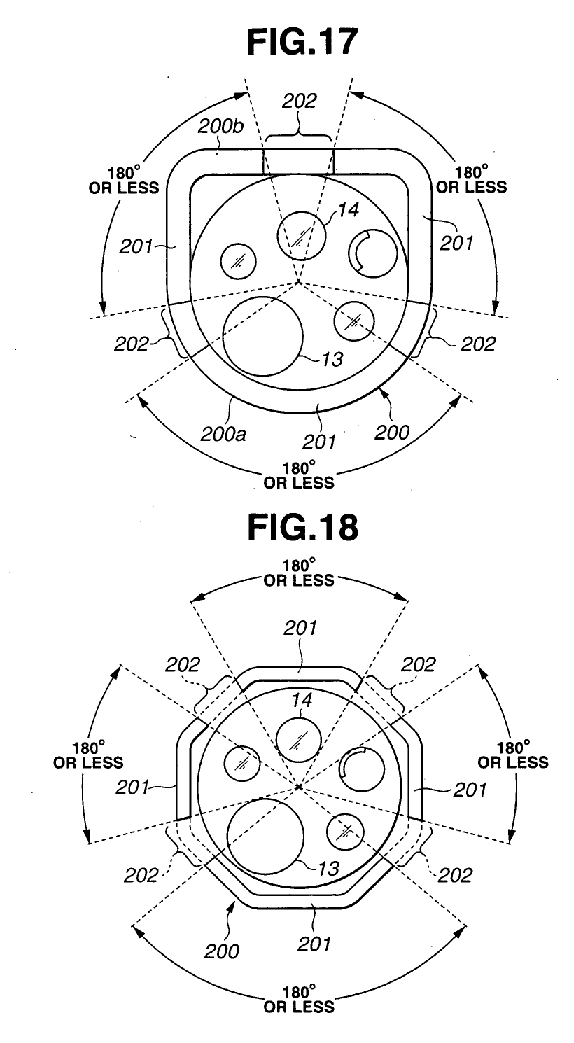

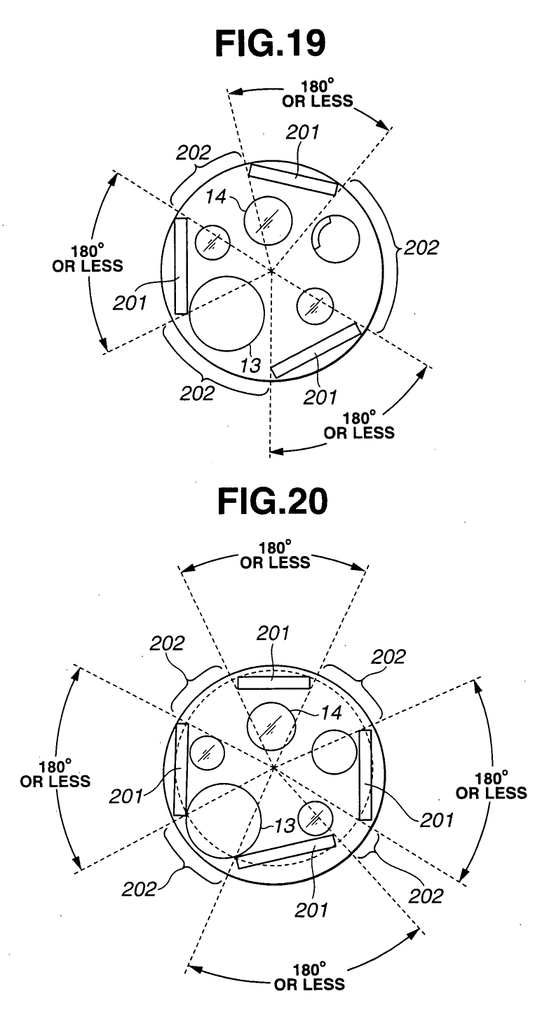

The protrusion of the distal hood component may have the shape shown in, for example, FIG. 17, FIG. 18, FIG. 19 and FIG. 20. The shape of the protrusion in each embodiment will be described below with reference to the drawings.

### EIGHTH EMBODIMENT

In the eighth embodiment shown in FIG. 17, the cross section of a protrusion 200 of a distal hood component is composed of a straight line portion 200a and an arc portion 200b, and concave portions 202 are provided at three places in order that the ranges of continuation of the convex portions 201 become 180° or less in the circumferential direction.

### NINTH EMBODIMENT

In the ninth embodiment shown in FIG. 18, the cross section of a protrusion 200 of a distal hood component is substantially in the shape of an octagon provided with concave portions 202 at four places in order that the ranges of continuation of the convex portions 201 become 180° or less in the circumferential direction.

### TENTH EMBODIMENT

In the tenth embodiment shown in FIG. 19, a distal hood component is integrally provided at the distal end of the endoscope while attachment and detachment cannot be performed. Three convex portions 201 in the shape of a straight line from the end surface of the endoscope distal end are substantially equidistantly provided along the perimeter circle of the endoscope distal end.

Consequently, the portions provided with no convex portion 201 constitute concave portions 202 and, thereby, the ranges of continuation of the convex portions 201 become 180° or less in the circumferential direction.

### ELEVENTH EMBODIMENT

In the eleventh embodiment shown in FIG. 20, four convex portions 201 in the shape of a straight line from the end surface of the endoscope distal end are provided.

In this case, all of the convex portions 201 are not necessarily provided along the perimeter circle of the endoscope distal end. As shown in the drawing, some convex portion 201 may be deviated from the outermost perimeter. The others are similar to those in the tenth embodiment shown in FIG. 19.

The embodiments of the present invention were described above. However, the present invention is not limited to the above-described embodiments, and as a matter of course, various modifications can be made within the spirit and scope of the present invention.

Industrial Applicability

According to the above-described present invention, the observational field of view can be easily ensured by the distal hood component, and an endoscope apparatus can be provided, which includes the distal hood component and has excellent observational performance.

Since the above-described protrusion can be deformed by application of a force from the end side thereof, application of an excessive force to the distal hood component can be prevented. Therefore, breakage of the distal hood component can be prevented from occurring and the durability can be improved, without using an expensive material. In addition, the patient can be prevented from having an uncomfortable feeling.

Furthermore, since the amount of the above-described protrusion visible in the observational field of view is increased when a part of the above-described protrusion is deformed, the surgeon can easily become aware that the distal hood component is deformed.

1. A distal hood component which is detachably or integrally provided at the distal end of an insertion portion of an endoscope and which comprises a protrusion protruding in the direction of the observational field of view of the endoscope,
wherein the protrusion is made of an elastically deformable soft material,
wherein at least two concave portions are provided in order to allow the ranges of continuation of convex portions of the protrusion to become about 180° or less in the circumferential direction, and
wherein the protrusion can be deformed by application of a force from the end side of the protrusion.

2. The distal hood component according to claim 1,
wherein the protrusion can be deformed by a force of about 0.29 MPa (3 kgf/cm²) or less applied from the end side of the protrusion.

3. The distal hood component according to claim 1,
wherein the protrusion is provided in order that the amount of the protrusion visible in the observational field of view is increased when a part of the protrusion is deformed.

4. The distal hood component according to claim 2,
wherein the protrusion is provided in order that the amount of the protrusion visible in the observational field of view is increased when a part of the protrusion is deformed.

5. The distal hood component according to claim 1,
wherein the protrusion can be deformed in an outward direction.

6. The distal hood component according to claim 2,
wherein the protrusion can be deformed in an outward direction.

7. A distal hood component comprising:
a protrusion which is made of an elastically deformable soft material and protrudes in the direction of the observation field of view of an endoscope from the distal end of an insertion portion of the endoscope,
wherein the protrusion is provided such that the ranges of continuation in the circumferential direction of the insertion portion becomes about 180° or less, and the protrusion can be deformed by application of a force in the direction of the observation field of view of the endoscope.

8. The distal hood component according to claim 7,
wherein the protrusion can be deformed by a force of about 0.29 MPa (3 kgf/cm²) or less applied from the end side of the protrusion.

9. The distal hood component according to claim 7,
wherein the protrusion is provided in order that the amount of the protrusion visible in the observational field of view is increased when a part of the protrusion is deformed.

10. The distal hood component according to claim 8,
wherein the protrusion is provided in order that the amount of the protrusion visible in the observational field of view is increased when a part of the protrusion is deformed.

11. The distal hood component according to claim 7,
wherein the protrusion can be deformed in an outward direction.

12. The distal hood component according to claim 8,
wherein the protrusion can be deformed in an outward direction.

13. A distal hood component comprising:
a protrusion which is made of an elastically deformable soft material and protrudes in the direction of the observation field of view of an endoscope from the distal end of an insertion portion of the endoscope; and
at least two concave portions provided in the protrusion, the amount of protrusion thereof in the direction of the observation field of view of the endoscope being smaller than that of the protrusion,
wherein the concave is provided such that the ranges of continuation of the protrusion thereof in the circumferential direction of the insertion portion becomes about 180° or less, and
the protrusion can be deformed by application of a force in the direction of the observation field of view of the endoscope.
